(19) United States
(12) Patent Application Publication
(10) Pub. No.: US 2022/0323790 A1
(43) Pub. Date: Oct. 13, 2022

(54) PARTICLE THERAPY SYSTEM, PARTICLE THERAPY METHOD, AND RECORDING MEDIUM

(71) Applicant: Hitachi, Ltd., Tokyo (JP)

(72) Inventors: Haruki ARITA, Tokyo (JP); Tomohisa IMAGAWA, Tokyo (JP); Naofumi ISHIGURO, Tokyo (JP)

(21) Appl. No.: 17/716,338

(22) Filed: Apr. 8, 2022

(30) Foreign Application Priority Data
Apr. 13, 2021 (JP) 2021-067687

(51) Int. Cl. A61N 5/10

(52) U.S. Cl. CPC A61N 5/1048; A61N 5/103; A61N 5/1079; A61N 2005/1087

(57) ABSTRACT

A particle therapy system includes: an accelerator that accelerates a particle beam; an irradiation system that applies the beam accelerated by the accelerator to a target; and a controller that controls the accelerator and the irradiation system. The controller has a treatment execution system that generates a control parameter which controls the accelerator and the irradiation system based on a treatment plan and has a treatment execution controller that controls the accelerator and the irradiation system based on the control parameter generated by the treatment execution system. When irradiation to the target by the irradiation system is suspended due to a failure or an artificial manipulation, the treatment execution system calculates an irradiated dose of the particle beam and an unirradiated dose of the particle beam, and the treatment execution system delivers at least the unirradiated dose to the treatment execution controller.

### CROSS-REFERENCE TO PRIOR APPLICATION

This application relates to and claim the benefit of priority from Japanese Patent Application No. 2021-067687 filed on Apr. 13, 2021 the entire disclosure of which is incorporated herein by reference.

### BACKGROUND

The present invention relates to a particle therapy system, a particle therapy method, and a recording medium.

The present invention relates to a particle therapy system suitable for particle therapy using a charged particle beam (ion beam), such as protons or heavy ions, and is a technique relating to a particle therapy system that is capable of continuing irradiation to an unirradiated volume when irradiation is temporarily suspended due to some reasons at the time of treating a certain patient and an operating method for the same.

As radiotherapy for cancer, particle therapy is known in which an ion beam of protons, heavy ions, or the like is applied onto the target volume of patient's cancer. When irradiation is stopped due to some reasons in performing particle therapy, there is a scheme that resumes irradiation to an unirradiated volume as disclosed in WO2016/121067.

As disclosed in WO2012/111125, there is an operating method that accomplishes irradiation to an unirradiated volume.

### SUMMARY

When a certain patient undergoes particle therapy, there is a prescription specific to the patient. This prescription is calculated and verified by a doctor using a treatment planning system, and irradiation is applied by a treatment execution system.

When irradiation is applied to a patient based on the prescription that is initially planned, the case is considered in which irradiation is suspended due to some reasons, such as a decision made by an operator, the detection of a failure in irradiation, or the detection of a failure in devices. At this time, the prescription that is initially planned has an irradiated volume and an unirradiated volume. In order to apply irradiation to the unirradiated volume, a prescription for unirradiated volumes is necessary (in the following, information on the irradiated volume is referred to as an actual irradiation result).

In the case in which it is necessary to generate a prescription for unirradiated volumes, there are a system in which a treatment planning system generates a prescription and a system in which a treatment execution system generates a prescription through no treatment planning system. In the case in which a treatment execution system generates a prescription for unirradiated volumes, it is necessary to assure that the generated prescription for unirradiated volumes matches the prescription of the treatment planning system when the actual irradiation result is added.

When treatment is resumed with the prescription for unirradiated volumes after suspension, since the prescription for unirradiated volumes is a prescription originally generated by the treatment execution system, from a device that monitors the treatment execution system in certain periods (in many cases, the treatment planning system), it seems as though a prescription different from the transmitted prescription is executed. This results in the detection of a failure in the treatment execution system, which sometimes leads to the suspension of irradiation. Therefore, in order to accomplish irradiation with the prescription for unirradiated volumes, the treatment execution system has to maintain the consistency of the state with relating devices.

The present invention is made in view of the problems, and an object is to provide a particle therapy system, a particle therapy method, and a recording medium that are capable of resuming treatment in a short time with the provision of consistency with the prescription of a treatment planning system.

In order to solve the problems, a particle therapy system according to an aspect of the present invention includes an accelerator that accelerates a particle beam, an irradiation system that applies the particle beam accelerated by the accelerator to a target, and a controller that controls the accelerator and the irradiation system. The controller has a treatment execution system that generates a control parameter which controls the accelerator and the irradiation system based on a treatment plan and has a treatment execution controller that controls the accelerator and the irradiation system based on the control parameter generated by the treatment execution system. When irradiation to the target by the irradiation system is suspended due to a failure or an artificial manipulation, the treatment execution system calculates an irradiated dose of the particle beam which has been already applied to the target and an unirradiated dose of the particle beam which is not applied to the target yet, and the treatment execution system delivers at least the unirradiated dose to the treatment execution controller.

According to the present invention, it is possible to resume treatment in a short time with the provision of consistency with the prescription of the treatment planning system.

### BRIEF DESCRIPTION OF THE DRAWINGS

FIG. 1 is a diagram showing an exemplary configuration of a particle therapy system of an embodiment;

FIG. 2 is a flowchart showing an example of the usual treatment of a particle therapy system;

FIG. 3 is a flowchart showing an example of a flow of suspension (up to suspension) due to a failure of the particle therapy system;

FIG. 4 is a flow showing an example of a resumption operation after suspension due to a failure (with no prescription for unirradiated volumes);

FIG. 5 is a flowchart showing an example of a resumption operation after suspension due to a failure (with a prescription for unirradiated volumes);

FIG. 6 is a diagram showing an example of the reception of a prescription and at the time of preparing irradiation by the particle therapy system of an embodiment;

FIG. 7 is a diagram showing an example of images during irradiation and at the time of suspension by the particle therapy system of an embodiment;

FIG. 8 is a diagram showing an example of images with a prescription for unirradiated volumes and at the time of preparing irradiation by the particle therapy system of an embodiment;

FIG. 9 is a diagram showing an example of images during irradiation of a prescription for unirradiated volumes by the particle therapy system of an embodiment; and

FIG. 10 is a diagram showing an example of images of the completion of irradiation of a prescription by the particle therapy system of an embodiment.

### DETAILED DESCRIPTION

In the following, an embodiment of the present invention will be described with reference to the drawings. Note that an embodiment that will be described below does not limit inventions in claims, and all components and all combinations of the components described in the embodiment are not necessarily required for solution of the inventions.

A particle therapy system according to the embodiment of the present invention is a particle therapy system using particle beams, such as a proton beam and a carbon beam. The particle beam used for the particle therapy system according to the present embodiment is non-limiting as long as the particle beam is a particle beam that is already practically used, such as the above-described proton beam and carbon beam, and a particle beam that will be practically used in future.

Note that in the case in which “data” is written in the present specification, the number of pieces of data is non-limiting, and its format is non-limiting. In addition, data and the like saved and stored in a storage medium in a so-called table format are also “data”, which is referred here.

The particle therapy system according to the present embodiment has a configuration below, for example.

In the case in which a system that monitors a treatment execution system has no information on a prescription for unirradiated volumes and no treatment is available, the treatment execution system has no match with prescription, many systems have an interlock that detects a failure, and thus a value that an actual irradiation result is added to the irradiation of the prescription for unirradiated volumes is transmitted to the monitoring system. However, since the treatment execution system that executes irradiation and a treatment execution controller operate based on the prescription for unirradiated volumes, the treatment execution system has to retain two types of pieces of data of a prescription and a prescription for unirradiated volumes in order to maintain consistency with relating systems.

It is necessary to assure that the generation of the prescription for unirradiated volumes calculated by the treatment execution system is appropriate to having been irradiated. When the treatment execution controller receives a prescription for unirradiated volumes, the prescription dose of the prescription for unirradiated volumes is added to an accumulating dose, for example, and the value is transmitted to the treatment execution system. After that, the prescription dose of the treatment planning system is matched with the prescription dose of the treatment execution system, and thus it can be said that the generation of the prescription for unirradiated volumes by the treatment execution system is appropriate.

According to the present embodiment, the treatment execution system generates the prescription for unirradiated volumes, and thus the resumption of treatment is made possible without through the treatment planning system, and treatment time is shortened. As main effects, shortening treatment time leads to an increase in the operation efficiency of a facility and a reduction in physical and mental stress of a treatment patient and the like.

In the case in which the treatment planning system generates a prescription for unirradiated volumes, it is necessary to transmit information on the actual irradiation result at the time of suspension of irradiation between the systems. The essential information that generates a prescription for unirradiated volumes is the dose of the actual irradiation result. Since radiation affects human bodies, the dose generally requires super high accuracy, and many significant digits have to be retained. A prescription for unirradiated volumes is generated in the treatment execution system, and thus round-off errors and the like due to the difference in data accuracy between the systems do not occur, and it is possible to generate a highly accurate prescription for unirradiated volumes.

In order to establish the interlock of the system that monitors the prescription of the treatment execution system during irradiation, there is also a scheme that transmits the original prescription from the treatment planning system. However, the prescription for unirradiated volumes calculated by the treatment execution system is added to the actual irradiation result and the added result is checked against the original prescription, which brings the effect of confirming the appropriateness of the arithmetic operation of generating the prescription for unirradiated volumes.

After the completion of irradiation, the treatment execution system transmits the actual result of the treatment to the treatment planning system. In the case in which the treatment planning system generates a prescription for unirradiated volumes at the time of suspension of irradiation, the treatment planning system finally has to add a partial actual result of the treatment with some method to compare the prescription for unirradiated volumes with the original prescription. However, the treatment execution system accomplishes irradiation using the prescription for unirradiated volumes, and the actual result of the treatment is transmitted as a group of pieces of information to the treatment planning system, which eliminates the addition of the actual result of the treatment by the treatment planning system.

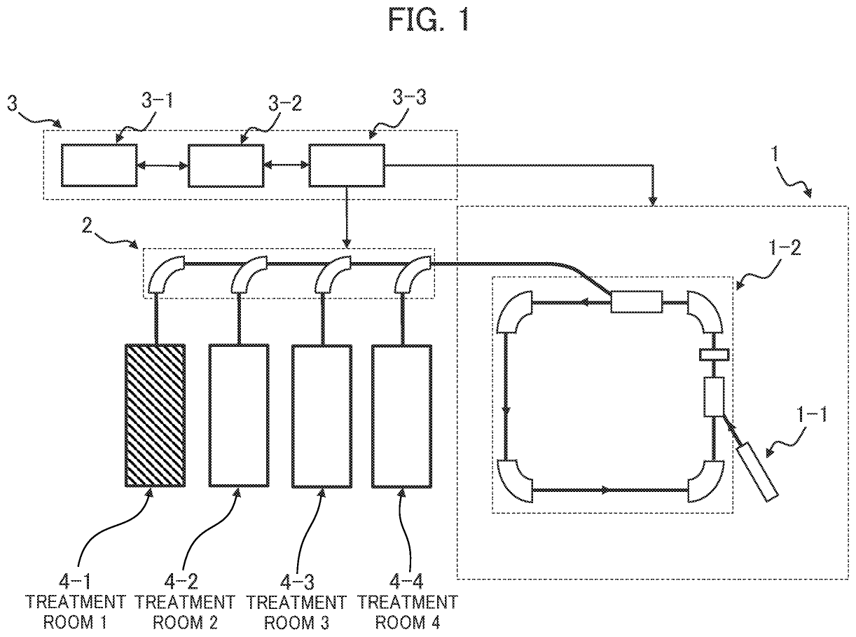

FIG. 1 is a schematic block diagram showing the particle therapy system of the embodiment.

As shown in FIG. 1, the particle therapy system according to the present embodiment includes an accelerator 1, four treatment rooms 4-1, 4-2, 4-3, and 4-4 (in the following, also written as the treatment room 4), a beam transport system 2 connected on the downstream side of the accelerator 1, and a general control system 3.

The accelerator 1 is a device that accelerates and emits an ion beam, and has an ion source (not shown), a preaccelerator (e.g., a linear accelerator) 1-1, and a synchrotron 1-2 as a circular accelerator. Note that instead of the synchrotron 1-2, for example, an accelerator having no preaccelerator, such as a cyclotron, or a linear accelerator may be used.

The preaccelerator 1-1 accelerates the energy of an ion beam generated in the ion source to the energy that is capable of entering the synchrotron 1-2. The ion beam accelerated at the preaccelerator 1-1 enters the synchrotron 1-2. After that, the ion beam having its energy accelerated to desired energy in the synchrotron 1-2 excites the electromagnet of the beam transport system 2 by the instructions of a treatment execution system 3-2 and a treatment execution controller 3-3 of the general control system 3, the ion beam is refracted and transported to the corresponding treatment rooms 4-1 to 4-4 that occupy the accelerator, and irradiation is achieved.

The treatment rooms 4-1 to 4-4 individually include an irradiation system (not shown) mounted on a rotating gantry (not shown) installed in the inside of the individual treatment rooms 4-1 to 4-4. The treatment rooms 4-1 to 4-4 are a first to a fourth treatment rooms for cancer patients, for example.

The irradiation system is a device that applies an ion beam, and includes two scanning electromagnets that independently scan beams in two directions orthogonal to a plane vertical to the orbit of the beam, a beam monitor, and the like.

Note that the case is described in which all the four treatment rooms have the same configuration. However, all the four treatment rooms do not necessarily have the same configuration, and a plurality of treatment rooms having separate configurations suited to treatments may be provided. For example, the irradiation system may be an irradiation system including a bending magnet, a scatterer system, a ring collimator, a patient collimator, a bolus, and the like, or may be an irradiation system including other configurations. The case is described in which the irradiation system is mounted on the rotating gantry in order to enable irradiation of a beam from a given direction to a target volume. However, the irradiation system may be fixed.

The general control system 3 is a control system that controls the accelerator 1, the beam transport system 2, and the irradiation system, and the treatment planning system 3-1, the treatment execution system 3-2, and the treatment execution system have the treatment execution controller 3-3.

The treatment planning system 3-1 has a storage unit in which a treatment plan generated by an operator is stored beforehand, and prior to starting the irradiation to the target volume (target) of a patient with a particle beam by the accelerator 1, the beam transport system 2, and the irradiation system, the treatment plan stored in the storage unit is delivered to the treatment execution system 3-2. The treatment planning system 3-1 monitors the control of particle beam irradiation by the treatment execution system 3-2 at regular time intervals.

The treatment execution system 3-2 generates a control parameter that controls devices constituting the beam transport system 2, the accelerator 1, and the treatment room 4 (the irradiation system in the treatment rooms, the devices in the beam transport system 2, the preaccelerator 1-1, and devices constituting the synchrotron 1-2) based on the treatment plan from the treatment planning system 3-1, and delivers the control parameter to the treatment execution controller 3-3.

The treatment execution controller 3-3 controls the devices constituting the beam transport system 2, the accelerator 1, and the treatment room 4 based on the control parameter delivered from the treatment execution system 3-2. The treatment execution controller 3-3 manages the actual result of irradiation with the particle beam by the irradiation system.

In the present embodiment, when a certain one of a plurality of the treatment rooms 4-1 to 4-4 once occupies the accelerator 1, the treatment execution system 3-2 of the general control system 3 controls the beam transport system 2 and the irradiation system such that the occupation of the accelerator 1 is maintained except when the occupation of the accelerator is intentionally cancelled or when a system failure occurs, (in the following, the control of maintaining the occupation of the accelerator 1 is also written as continuous multi-field irradiation control).

For example, when a certain patient undergoes treatment in a certain one of the treatment rooms 4-1, 4-2, 4-3, and 4-4, the treatment execution system 3-2 performs continuous multi-field irradiation control until all the treatment prescribed to the certain patient is finished.

Alternatively, when a certain one of the treatment rooms 4-1, 4-2, 4-3, and 4-4 once occupies the accelerator 1 for irradiation for treatment based on the selection by a therapist who applies particle therapy to a patient, the treatment execution system 3-2 performs continuous multi-field irradiation control until all of predetermined irradiation is finished.

Alternatively, when a certain one of the plurality of the treatment rooms 4-1 to 4-4 occupies the accelerator 1, the treatment execution system 3-2 performs continuous multi-field irradiation control such that the occupation of the accelerator by the specific one of treatment rooms 4-1, 4-2, 4-3, and 4-4 is maintained.

Alternatively, when a specific prescription is applied to a patient in a non-specific one of the treatment rooms 4-1, 4-2, 4-3, and 4-4, the treatment execution system 3-2 performs continuous multi-field irradiation control until all the treatment of the specific prescription is finished.

Note that when control that is not continuous multi-field irradiation control is selected, the treatment execution system 3-2 performs discontinuous multi-field irradiation control. For discontinuous multi-field irradiation control at this time, publicly known control, such as the occupation of the accelerator on the first-come first-served basis and the occupation of the accelerator based on the priority level.

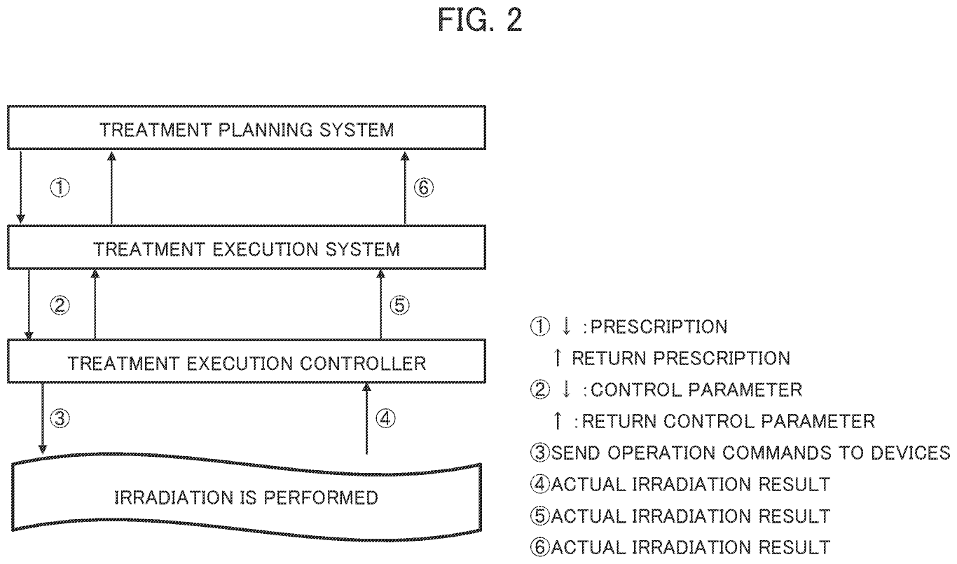

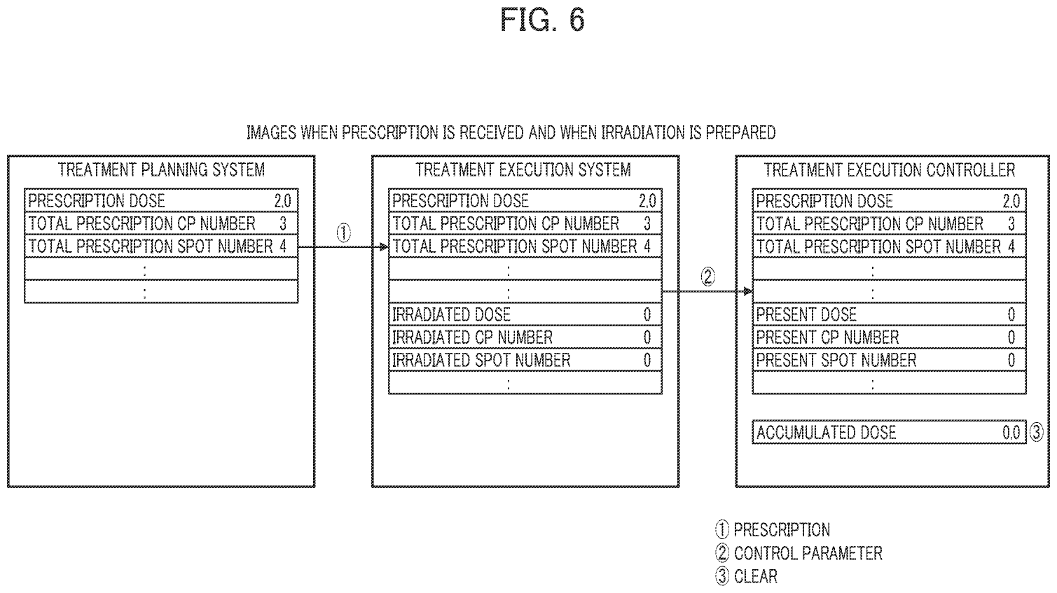

Next, referring to FIGS. 2 and 6, a data flow between the treatment planning system 3-1, the treatment execution system 3-2, and the treatment execution controller 3-3 will be described.

The treatment planning system 3-1 generates a prescription specific to a patient, and transmits the irradiation of the prescription to be performed to the treatment execution system 3-2. The treatment execution system 3-2 that receives the prescription transmits the control parameter for achieving the irradiation of the prescription to the treatment execution controller 3-3.

At this time, as a method of ensuring whether the prescription is correctly transmitted, the control parameter received by the treatment execution controller 3-3 is returned to the treatment execution system 3-2, and the treatment execution system 3-2 checks the consistency of the transmitted control parameter with the returned control parameter.

It is a general operation of the system that the treatment execution system 3-2 converts the prescription into a prescription format, which can be recognized by the treatment planning system 3-1, based on the control parameter returned from the treatment execution controller 3-3, and returns the prescription for checking the consistency of the transmitted prescription with the returned prescription by the treatment planning system 3-1. An example of a general system in which when a failure is detected in any check of consistency, there is an interlock that is incapable of starting irradiation.

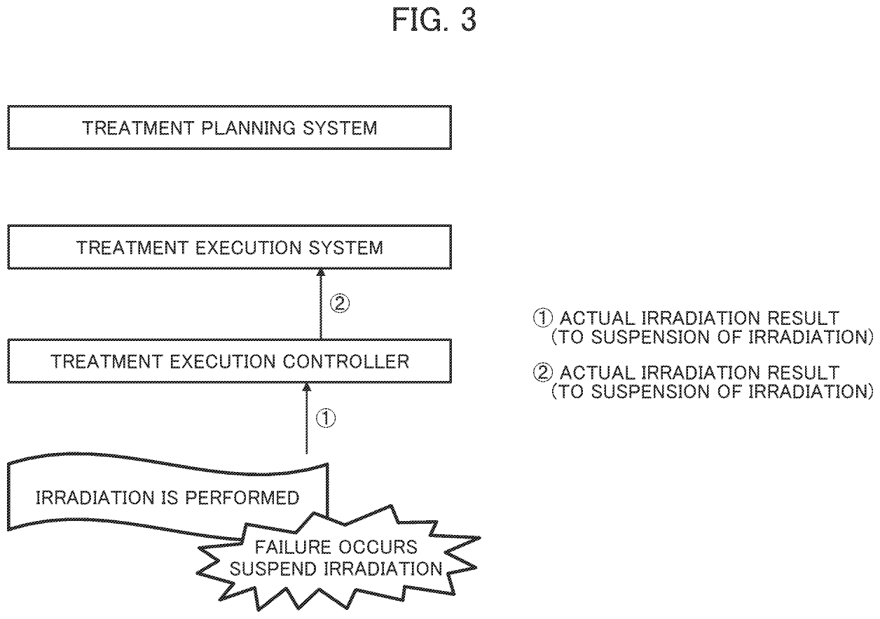

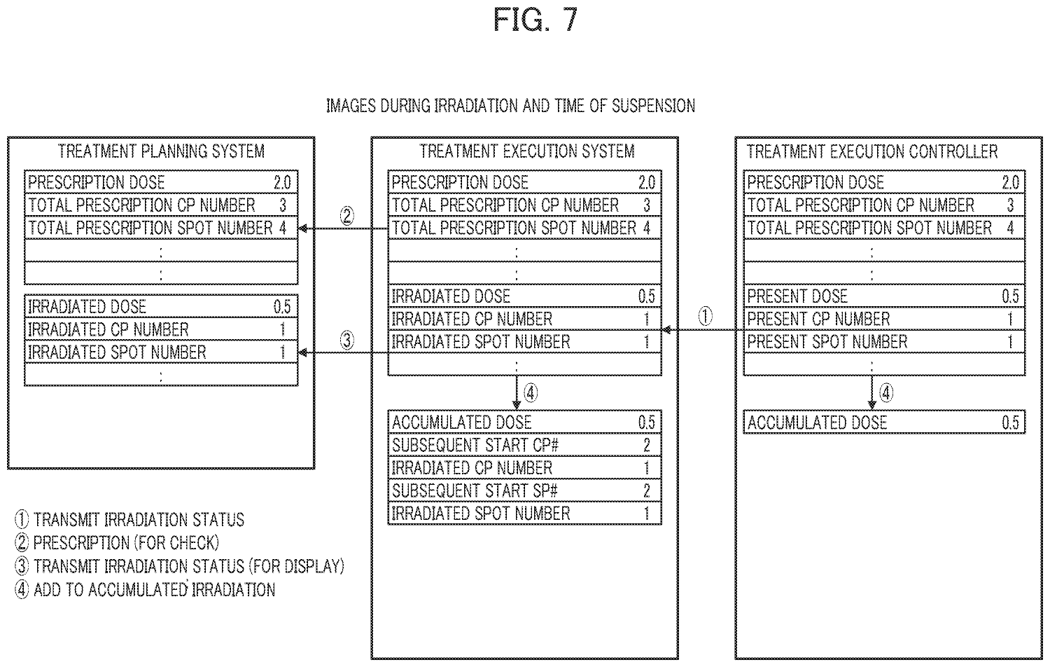

As shown in FIGS. 3 and 7, when some kind of failure is detected during irradiation, the irradiation is sometimes stopped. It is sometimes difficult to immediately resume irradiation depending on the content of the failure. At this time, the case is considered in which a patient is temporarily evacuated, and in a facility in which there are other treatment rooms 4-1 to 4-4, the right of occupation of the accelerator 1 is moved to another treatment room.

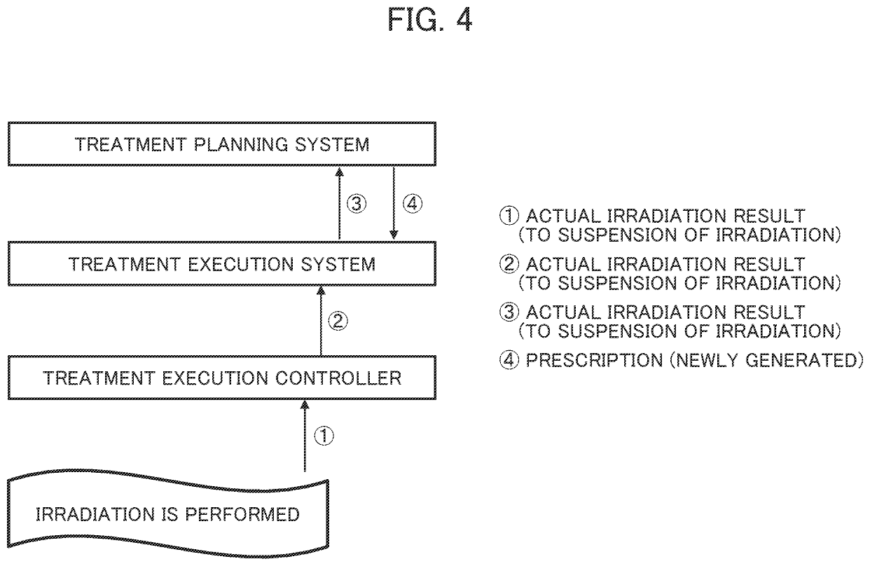

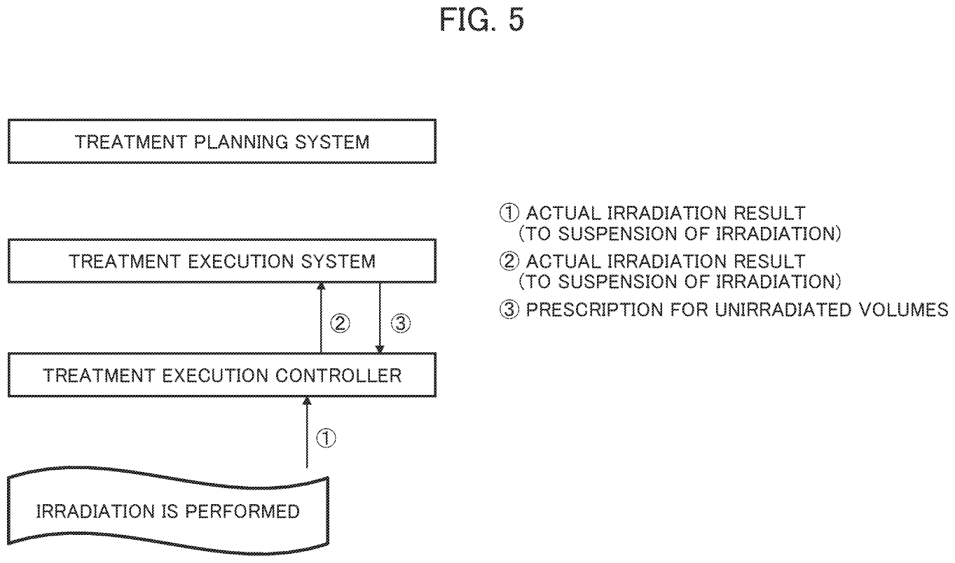

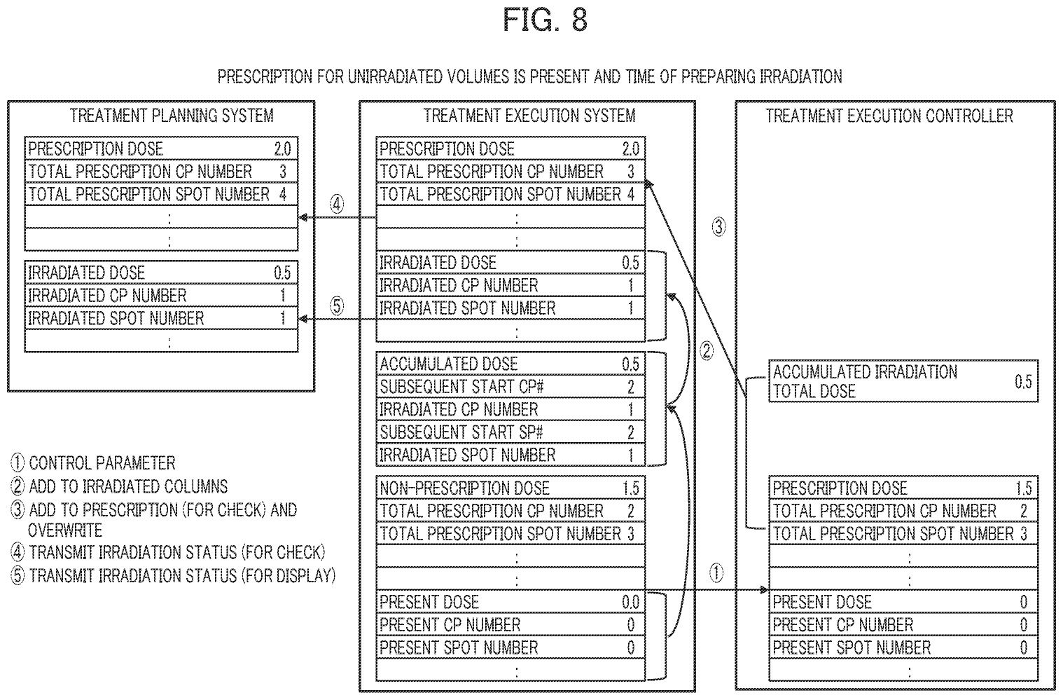

As described above, the accelerator 1 is often shared by the plurality of the treatment rooms 4-1 to 4-4. When new irradiation is started in the other treatment rooms 4-1 to 4-4, a part of the above-described control parameter is discarded (see FIG. 4). Therefore, in the case in which an unirradiated volume is irradiated, a new control parameter has to be again transmitted to the treatment execution controller 3-3 (see FIGS. 5 and 8).

The treatment execution system 3-2 generates this prescription for unirradiated volumes that is the basis for the new control parameter, and thus it is possible to achieve the planned irradiation of the prescription without generating a new prescription by the treatment planning system 3-1.

Here, since the prescription for unirradiated volumes is a prescription that is not present in the treatment planning system 3-1, generally, it is not possible to start irradiation due to the interlocks (see Circled number 1 and Circled number 2 in FIG. 2) (see FIG. 8).

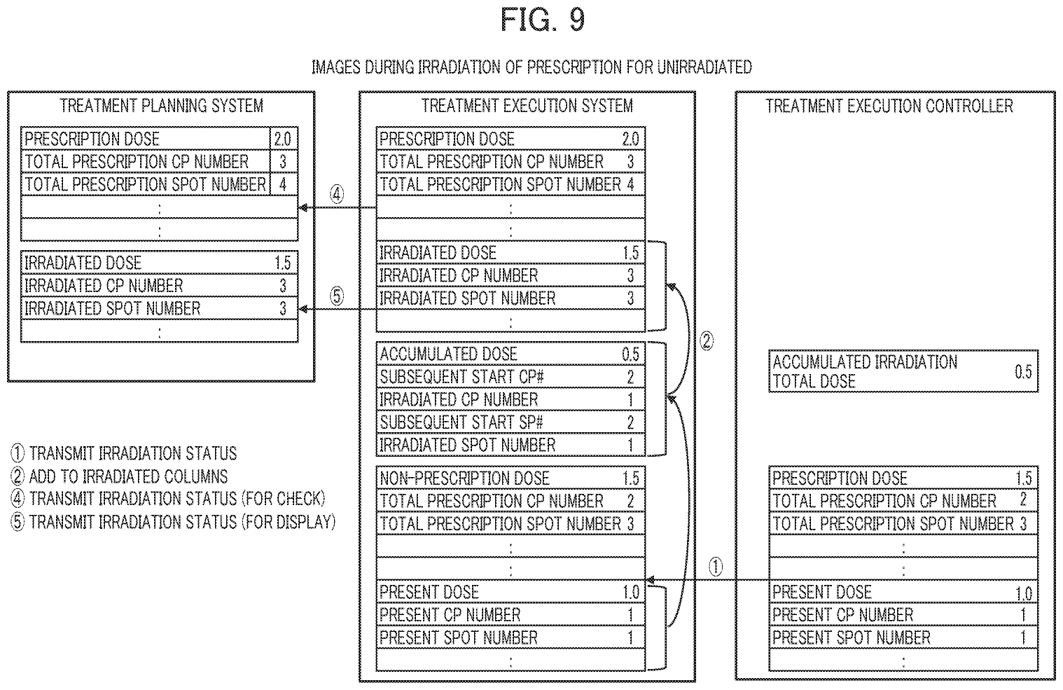

Therefore, the treatment execution system 3-2 transmits the content that the actual irradiation result is added with the prescription for unirradiated volumes as a prescription to the treatment planning system 3-1. As a result, the interlock due to the treatment planning system 3-1 is eliminated, and simultaneously, the prescription is compared with the content of the actual irradiation result and the prescription for unirradiated volumes, and thus the consistency of the prescription for unirradiated volumes generated by the treatment execution system is also ensured (see FIG. 9). Under the situations during irradiation of the prescription for unirradiated volumes, the treatment execution system performs operation while retaining two types of data to be transmitted to the treatment planning system and data actually having been transmitted to the treatment execution controller in order to operate the irradiation of the prescription for unirradiated volumes, and thus it is possible to achieve the irradiation of the prescription for unirradiated volumes.

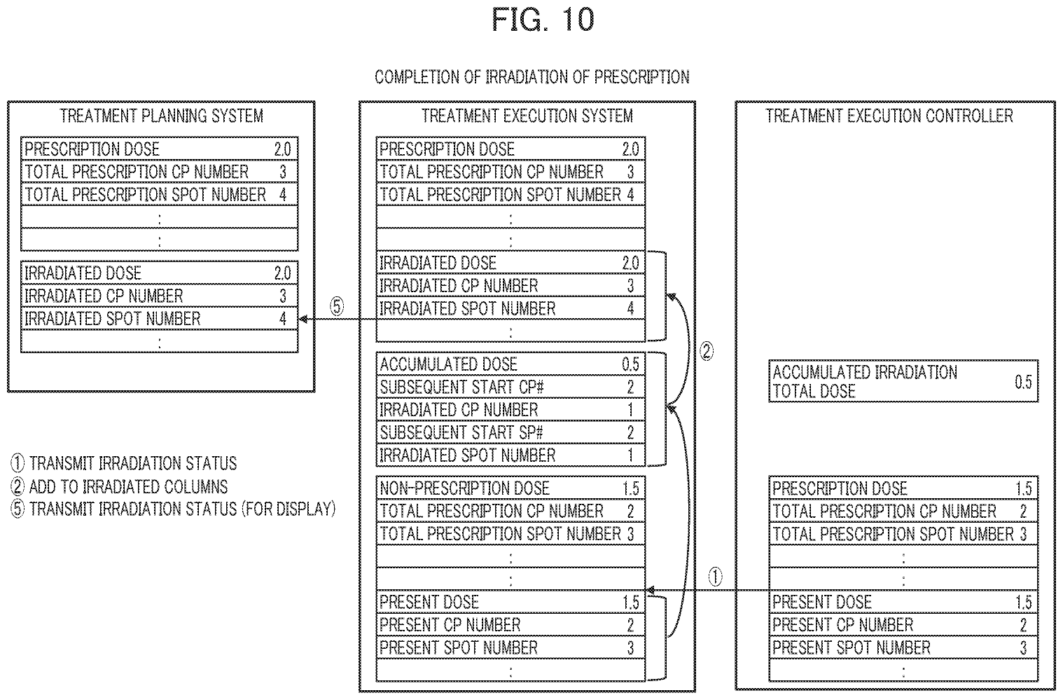

As shown in FIG. 10, generally, as the operation when the treatment execution system 3-2 and the treatment execution controller 3-3 complete the irradiation that satisfies the prescription of the treatment planning system 3-1, the treatment execution system 3-2 collects the actual irradiation result from the treatment execution controller 3-3, transmits the actual irradiation result to the treatment planning system 3-1, and the treatment planning system 3-1 compares the prescription with the actual irradiation result for recording.

When the prescription for unirradiated volumes is performed, since the actual irradiation result collected by the treatment execution controller 3-3 is different from the prescription transmitted from the treatment planning system 3-1, the treatment execution controller 3-3 adds the collected actual irradiation result to the old actual irradiation result used for generating the prescription for unirradiated volumes, and thus the actual irradiation result corresponding to the prescription of the treatment planning system 3-1 is generated. Therefore, the treatment execution system 3-2 can transmit an actual irradiation result with no defect to the prescription of the treatment planning system 3-1.

Consequently, according to the present embodiment, it is possible to resume treatment in a short time with the provision of consistency with the prescription of the treatment planning system.

### EXEMPLARY MODIFICATIONS

Note that the present invention is nonlimiting to the foregoing embodiment, and includes various exemplary modifications. For example, the foregoing embodiment is described in detail for easily understanding the present invention, and is not necessarily limited to ones including all the described configurations. Further, a part of the configuration of an embodiment can be replaced with the configuration of another embodiment, and the configuration of an embodiment can be added with the configuration of another embodiment as well. Moreover, in regard to a part of the configuration of the embodiments, another configuration can be added, removed, and replaced.

As an example, a configuration may be provided in which when irradiation to a target by the irradiation system is suspended due to a failure or an artificial manipulation, the treatment execution system 3-2 maintains the occupation of the accelerator 1 by the treatment rooms 4-1 to 4-4 in which the irradiation system is disposed until the irradiation system that is suspending irradiation resumes irradiation based on the prescription for unirradiated volumes or the treatment execution system 3-2 selects whether to permit another treatment room to occupy the accelerator 1.

Various parameters shown in FIGS. 6 to 10 may be displayed by a display device of the treatment execution system 3-2, the treatment execution controller 3-3, or the treatment planning system 3-1.

A part of or all the configurations, functions, processing units, processing sections, and the like may be implemented with hardware by design using integrated circuits. The configurations, functions, and the like may be implemented with software by a processor interpreting and executing programs that implement these functions. Information on programs, tables, files, and the like can be placed on a recording device, such as a memory, hard disk, and SSD, or on a recording medium, such as an IC card, SD card, and DVD.

Control lines and information lines that are considered to be necessary for description are shown, and all control lines and information lines for a product are not necessarily shown. Actually, it may be considered that almost all the configurations are connected to each other.

What is claimed is:

1. A particle therapy system comprising:
an accelerator that accelerates a particle beam;
an irradiation system that applies the particle beam accelerated by the accelerator to a target; and
a controller that controls the accelerator and the irradiation system,
wherein: the controller has a treatment execution system that generates a control parameter which controls the accelerator and the irradiation system based on a treatment plan and has a treatment execution controller that controls the accelerator and the irradiation system based on the control parameter generated by the treatment execution system; and
when irradiation to the target by the irradiation system is suspended due to a failure or an artificial manipulation, the treatment execution system calculates an irradiated dose of the particle beam which has been already applied to the target and an unirradiated dose of the particle beam which is not applied to the target yet, and the treatment execution system delivers at least the unirradiated dose to the treatment execution controller.

2. The particle therapy system according to claim 1,
wherein the treatment execution system calculates the irradiated dose and the unirradiated dose based on information on irradiation status of the particle beam delivered from the treatment execution controller.

3. The particle therapy system according to claim 2,
wherein the treatment execution system delivers the irradiated dose and the unirradiated dose to a treatment planning system that generates the treatment plan and monitors execution of the treatment plan.

4. The particle therapy system according to claim 2,
wherein after completion of irradiation of the particle beam to the target by the irradiation system, the treatment execution system delivers the irradiated dose of the particle beam before suspending irradiation of the particle beam and the irradiated dose of the particle beam after suspending irradiation of the particle beam to a treatment planning system that generates the treatment plan and monitors execution of the treatment plan.

5. The particle therapy system according to claim 1, comprising
a plurality of treatment rooms in which the irradiation system is disposed,
wherein: when irradiation to the target by the irradiation system is suspended due to a failure or an artificial manipulation, until the irradiation system in which irradiation is being suspended resumes irradiation based on the unirradiated dose, the treatment execution system maintains occupation of the accelerator by the treatment room in which the irradiation system is disposed or selects whether to permit another treatment room to occupy the accelerator.

6. A particle therapy method by a particle therapy system including an accelerator that accelerates a particle beam, an irradiation system that applies the particle beam accelerated by the accelerator to a target, and a controller that controls the accelerator and the irradiation system, the controller having a treatment execution system that generates a control parameter which controls the accelerator and the irradiation system based on a treatment plan and a treatment execution controller that controls the accelerator and the irradiation system based on the control parameter generated by the treatment execution system, the method comprising
calculating, when irradiation to the target by the irradiation system is suspended due to a failure or an artificial manipulation, an irradiated dose of the particle beam which has been already applied to the target and an unirradiated dose of the particle beam which is not applied to the target yet, and delivering at least the unirradiated dose to the treatment execution controller.

7. A recording medium recording a computer program executed by a computer that controls a particle therapy system including an accelerator that accelerates a particle beam, an irradiation system that applies the particle beam accelerated by the accelerator to a target, and a treatment execution controller that controls the accelerator and the irradiation system based on a control parameter, the computer program causing the computer to implement a function of
calculating, when irradiation to the target by the irradiation system is suspended due to a failure or an artificial manipulation, an irradiated dose of the particle beam which has been already applied to the target and an unirradiated dose of the particle beam which is not applied to the target yet, and delivering at least the unirradiated dose to the treatment execution controller.
